(19) United States
(12) Patent Application Publication
(10) Pub. No.: US 2026/0094677 A1
(43) Pub. Date: Apr. 2, 2026

(54) METHODS OF PREDICTING PROPERTIES OF A CHEMICAL SYSTEM USING SURROGATE MODELS

(71) Applicant: CAMERON INTERNATIONAL CORPORATION, Houston, TX (US)

(72) Inventors: Sergey MAKARYCHEV-MIKHAILOV, Cambridge (GB); Jesse FARRELL, Sugar Land, TX (US)

(21) Appl. No.: 19/112,646

(22) PCT Filed: Sep. 19, 2023
(86) PCT No.: PCT/US2023/033157

§ 371 Date: Mar. 18, 2025

Related U.S. Application Data
(60) Provisional application No. 63/376,169, filed on Sep. 19, 2022.

(51) Int. Cl. G16C 20/30; G06N 20/00; G16C 20/70

(52) U.S. Cl. CPC G16C 20/30; G06N 20/00; G16C 20/70

(57) ABSTRACT

Methods of predicting physicochemical properties of a chemical system using a family of surrogate or reduced order models, trained on first principle simulation results. The models are created using machine learning techniques. The chemical system can be a complex multicomponent and multiphase system such as produced water.

### CROSS-REFERENCE TO RELATED APPLICATIONS

Any and all applications for which a foreign or domestic priority claim is identified in the Application Data Sheet as filed with the present application are hereby incorporated by reference under 37 CFR 1.57. The present application claims priority benefit of U.S. Provisional Application No. 63/376,169, filed Sep. 19, 2022, the entirety of which is incorporated by reference herein and should be considered part of this specification.

### BACKGROUND

### Field

The present disclosure relates generally to methods of predicting a broad range of physicochemical properties of a complex chemical system and, more specifically, to such a method using reduced order models (ROM), also known as surrogate models.

### Description of the Related Art

Water is prolific in the E&P (oil and gas exploration and production) industry, and produced water is one of the largest, or the largest, waste streams in the industry. Produced water properties affect and are important for flow assurance, 3-phase flow PVT modeling, and fluid compatibility purposes across well construction, stimulation, and production operations. Time and labor intensive lab tests and/or sophisticated simulators are required to determine water physicochemical properties, and to understand their dependence on external parameters, such as temperature, pressure, mixing with water coming from other sources, changes due to chemical additions, etc.

Simulators available to the industry usually rely on first principle thermodynamic calculations. The inputs include water composition, e.g., concentrations of dissolved species, sometimes, in contact with minerals and gases, and conditions, such as temperature and pressure. The calculations use thermodynamic constants, equilibrium constants, e.g., solubility products measured at different conditions, and are performed using various theoretical models, which quantify interactions between dissolved species, for example, Debye-Hückel, Pitzer, Raoult and other equations. Computations are often done iteratively and depending on complexity of the simulated system, can take some time, particularly when variations of input parameters are required. Importantly, the simulation results are only valid within a range of conditions, where reliable constant values are available, approximations for the applied equations are reasonable, and the resulting thermodynamic models are calibrated.

First principle thermodynamic models are usually available as stand-alone applications, and while their incorporation into digital workflows is generally possible, it is often not an easy task. For example, one such simulator, ScaleSoftPitzer, developed by the Brine Consortium at Rice University, is distributed (to Consortium members) as an Excel file with the Visual Basic for Applications (VBA) code. Extracting this code and translating it to more practical and appropriate programming languages is a challenge.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

### SUMMARY

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

The disclosed techniques are directed to a method for predicting physicochemical properties of complex chemical systems using a reduced order model (ROM). The ROM can be trained on first principle thermodynamic simulations. The method can provide prediction of a plurality of physicochemical properties. The complex chemical system can be produced water. The physicochemical properties, e.g., water properties, can include density, thermal conductivity, heat capacity, and/or scaling potential for scale-forming minerals.

In some configurations, a method of predicting physicochemical properties of a chemical system includes training and optimizing reduced order models (ROMs) for one or more target properties using one or more machine learning models, and predicting the physicochemical properties of the chemical system using the trained and optimized ROMs.

The method can further include screening multiple machine learning models for the one or more target properties using model metrics to compare models, and selecting one or more of the machine learning models based on the model metrics. Training and optimizing the ROMs can include training and optimizing the ROMs using the one or more machine learning models selected based on the model metrics.

The chemical system can be or include produced water. The method can include performing principal component analysis on a representative dataset to produce an engineered dataset. The method can further include using the engineered dataset to train the ROMs. The method can include deploying the trained and optimized ROMs in one or more digital workflows. The one or more digital workflows can include optimizing one or more processes. Such processes can include one or more of dosing of a chemical, adjustment of a pump flow rate, regulation of a pressure, adjustment of fluid temperature within equipment, and actuating a valve.

Various refinements of the features noted above may exist in relation to various aspects of the present disclosure. Further features may also be incorporated in these various aspects as well. These refinements and additional features may exist individually or in any combination. For instance, various features discussed below in relation to one or more of the illustrated embodiments may be incorporated into any of the above-described aspects of the present disclosure alone or in any combination. The brief summary presented above is intended only to familiarize the reader with certain aspects and contexts of embodiments of the present disclosure without limitation to the claimed subject matter.

### BRIEF DESCRIPTION OF THE DRAWINGS

Various aspects of this disclosure may be better understood upon reading the following detailed description and upon reference to the drawings in which:

FIG. 1 graphically illustrates results of principal component analysis on an enriched produced water dataset; FIG. 1A illustrates a PCA score plot, as PC1 (74.5% explained variance) and PC2 (8.4% explained variance); FIG. 1B illustrates explained variance as a function of number of principal components;

FIG. 2 illustrates a PCA loadings table;

FIG. 3 illustrates histograms of original features in the training dataset; and

FIG. 4 illustrates a prototype of a web-based water properties and scale potential simulator according to embodiments of the disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and enterprise-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Water properties are important for well construction, stimulation, and production operations in the oil and gas industry. Time and labor intensive lab tests and/or sophisticated simulators are required to determine water physicochemical properties, and to understand their dependence on external parameters, such as temperature, pressure, mixing with water coming from other sources, and changes due to chemical additions. Simulators available to the industry usually rely on first principle thermodynamic calculations and are performed using various theoretical models. Computations are often done iteratively and depending on complexity of the simulated system, can take some time, particularly when variations of input parameters are required. Importantly, the simulation results are only valid within a range of conditions, where reliable constant values are available, approximations for the applied equations are reasonable, and the resulting thermodynamic models are calibrated. Such first principle thermodynamic models are often difficult to incorporate into digital workflows.

Using surrogate or reduced order models is a well-established approach, which provides fast solution to complex computational problems. Theoretical physics problems, where solving large systems of partial differential equations (PDE) is required, or modeling complex industrial processes with many empirical parameters, have become common applications of surrogate models.

However, multiple input parameters, the intrinsic system complexity and many required output parameters make the simulations of complex chemical systems with ROMs particularly difficult. The present disclosure provides methods to predict physicochemical properties of complex chemical systems, such as in the oilfield industry and other industries, using reduced order models. Such methods can enable complex multicomponent and multiphase systems, such as produced water, to be described with ROM with reasonable accuracy.

The following examples outline the development of ROMs, for example, for predicting physicochemical properties. While the following examples outline the development of ROMs for predicting physicochemical properties of reduced water, the methods described herein can be used to develop ROMs for predicting the physicochemical properties of other complex chemical systems.

### Example 1. Representative Produced Water Dataset

In this example, the publicly available USGS Produced Water Database ver. 2.3 was used to define the feature space for the ROM training dataset. The database contains information on ˜115,000 water samples collected from different oil and gas reservoirs in the United States since 1905. The original data was cleaned with poorly populated or inconsistent outlier samples being removed, and ion/species concentrations converted to the same units, as mass percent.

Fourteen key produced water ions, which constitute over 99.5% of water solutes were defined, as:
a. Cations: Na⁺, K⁺, Mg²⁺, Ca²⁺, Sr²⁺, Ba²⁺, Fe²⁺, Zn²⁺
b. Anions: Cl⁻, HCO₃⁻, SO₄²⁻, Br⁻, F⁻ and H₂S (the latter is reported as non-ionic but can dissociate in water and at produced water pH to form primarily HS⁻ ions).

Data enrichment was performed imputing missing values of water density (required for concentration unit conversions), and ion concentrations of minor ions, which are either always (K⁺, Mg²⁺, Br⁻), or sometimes (Sr²⁺, Ba²⁺, Fe²⁺, Zn²⁺, F⁻, and H₂S) present, were performed using the Random Forest machine learning algorithm, using concentrations of core ions (Nat, Ca²⁺, Cl⁻, HCO₃⁻, and SO₄⁻) and density as inputs. In embodiments, the rare ions might be added to a randomly selected fraction of samples, such as Sr²⁺: to 80% of samples, Ba²⁺: 25%, Fe²⁺: 50%, Zn²⁺: 25%, F⁻: 20%, H₂S: 25%

Of note, many samples in the dataset are obviously oversaturated in respect to certain salts, like calcium carbonate, calcium sulfate, or even sodium chloride. This is an indication that many samples were characterized under non-equilibrium conditions, and so are likely realistic to high temperature and pressure environment.

The resulting clean and enriched dataset with ˜85,000 samples was then checked against a smaller dataset of produced waters, collected in various places around the world, and was found to be representative covering the whole range of real-life produced water compositions. Statistics of the resulting dataset are shown in Table 1 below. Any new produced water samples are expected to fall within the ranges covered by this dataset with very high probability.

To ensure that charge balance of all ions present in water sample solutions is equal to zero (meaning there is no non-physical excess of either cations or anions), concentrations of all ions can be recalculated to concentrations of their corresponding sodium or chloride salts with necessary additions of Na⁺ or Cl⁻ ions.

TABLE 1
Statistics of ion concentrations in the enriched dataset, mass %.

| Ion | Mean | StDev | Median | 99% | Max |
|---|---|---|---|---|---|
| Na | 2.57 | 2.57 | 1.6 | 9.4 | 12.2 |
| K | 0.065 | 0.100 | 0.03 | 0.47 | 1.74 |
| Mg | 0.081 | 0.115 | 0.03 | 0.53 | 1.15 |
| Ca | 0.49 | 0.76 | 0.15 | 3.7 | 6.1 |
| Sr | 0.021 | 0.036 | 0.004 | 0.162 | 0.823 |
| Ba | 0.004 | 0.019 | <10⁻⁶ | 0.060 | 0.893 |
| Fe | 0.0034 | 0.0081 | 0.0009 | 0.0300 | 0.297 |
| Zn | 0.0001 | 0.0007 | 0.0000 | 0.0021 | 0.030 |
| Cl | 4.98 | 5.23 | 2.88 | 17.5 | 20.7 |
| HCO₃ | 0.076 | 0.137 | 0.033 | 0.67 | 1.75 |
| SO₄ | 0.093 | 0.150 | 0.030 | 0.73 | 1.32 |
| F | 0.0001 | 0.0004 | 0.0000 | 0.0013 | 0.0259 |
| H₂S | 0.0025 | 0.0066 | 0.0000 | 0.0319 | 0.1098 |
| Br | 0.0252 | 0.0417 | 0.0099 | 0.2090 | 0.5426 |

### Example 2. Engineered Produced Water Dataset

As the dataset described in Example 1 might still be too large for machine learning applications, a reduced dataset was created with 10,000 samples, preserving the representativeness of the original dataset (of Example 1). As water ions concentrations are not independent of each other (and over 80% of dissolved ions constitute just NaCl), selection of representative samples can be performed on non-correlated dataset features. Principal component analysis (PCA) can be used. In the present example, PCA based on the original features (ion concentrations) generated their linear combinations, principal components (PC), which are orthogonal to each other and explain most variance in the original data.

Before performing PCA, concentrations of all ions can be min-max normalized, using the formula:

$${x}_{scaled} = \frac{x {x}_{min}}{{x}_{max} - {x}_{min}} \qquad ( 1 )$$

While other scaling algorithms can also be used, such normalization allows to scale all ion concentrations equally, as all now have the range between 0 and 1, preventing bias towards features with high absolute values.

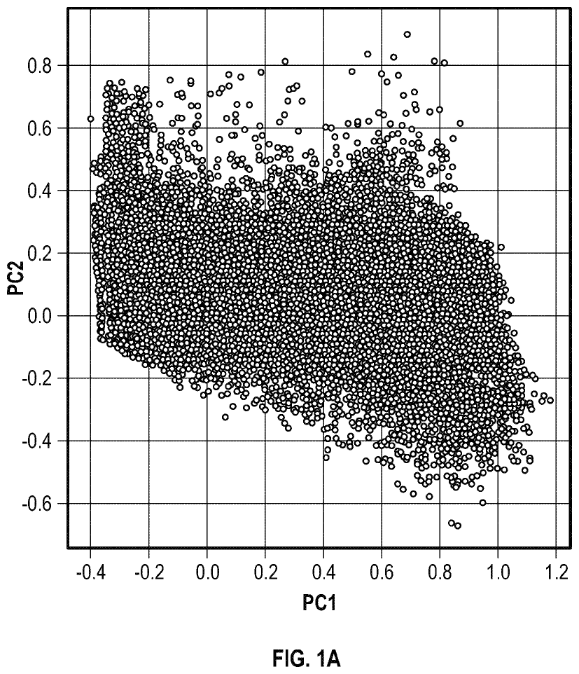

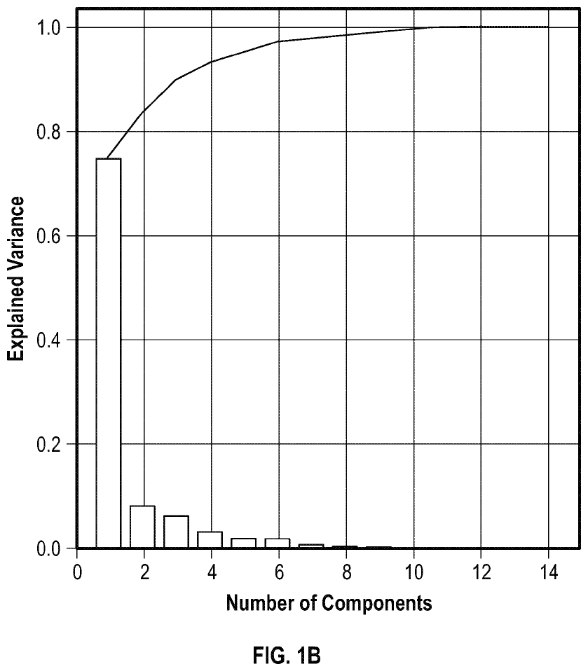

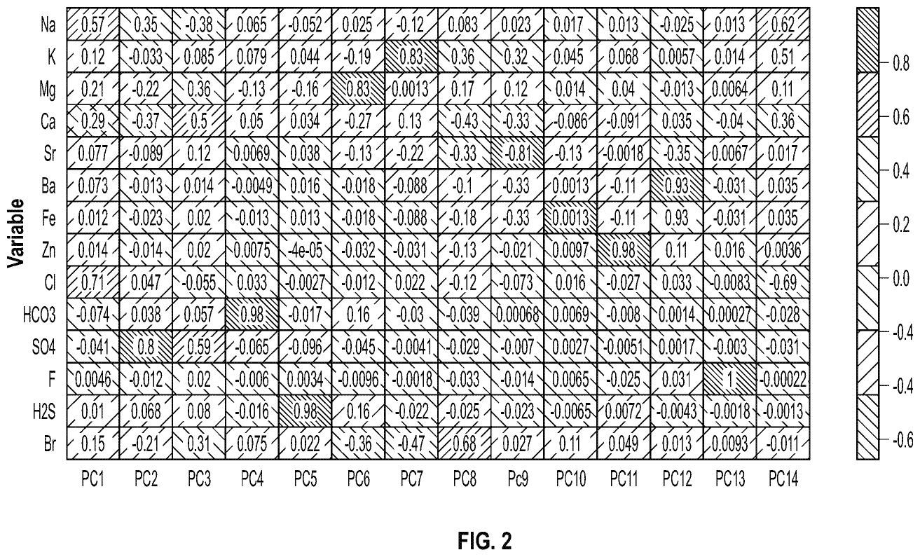

The PCA results on the enriched dataset are presented in FIGS. 1A-1B and FIG. 2. One observation (viewing FIG. 1A) is that the dataset is free from obvious multidimensional outliers, due to the data cleaning procedures applied in the previous step. Second, while the number of calculated PCs is equal to the number of ions in the dataset, ten PCs explain over 99% of variance in the data (as indicated in FIG. 1B). This illustrates how PCA can be used to reduce data dimensionality.

FIG. 2 illustrates a PCA loadings table. As shown, loadings of the first components are correlated with major ions, but minor ions still contribute even PCs up to 13; however, PC14 is redundant (as depending mostly on Na⁺ and Cl⁻).

In some configurations, once the new PC feature space is constructed, the sampling can be performed by sorting water samples by one PC value at a time, picking, for example, 800 samples evenly spread across that PC, and repeating the procedure for PCs from 1 to 13. Duplicated samples can then be removed.

The resulting reduced produced water dataset is representative of the original cleaned dataset. A few additional samples, which might include seawaters of variable composition and pure salt solutions (brines), can be manually added to the dataset for completeness.

In some configurations of the disclosure, the prediction models for certain properties of interest might benefit from derivative features built on original ones. For example, in predicting calcium sulphate scales (e.g., anhydrite, CaSO₄, or gypsum, CaSO₄·2H₂O), those skilled in the art of chemistry can anticipate that in addition Ca²⁻ and SO₄²⁻ ion concentrations, their product ([Ca²⁺]·[SO₄²⁻]) might also be important. Such products, as engineered features, can be calculated for all combinations of ions present in key oilfield scales and added to the enriched dataset as new features.

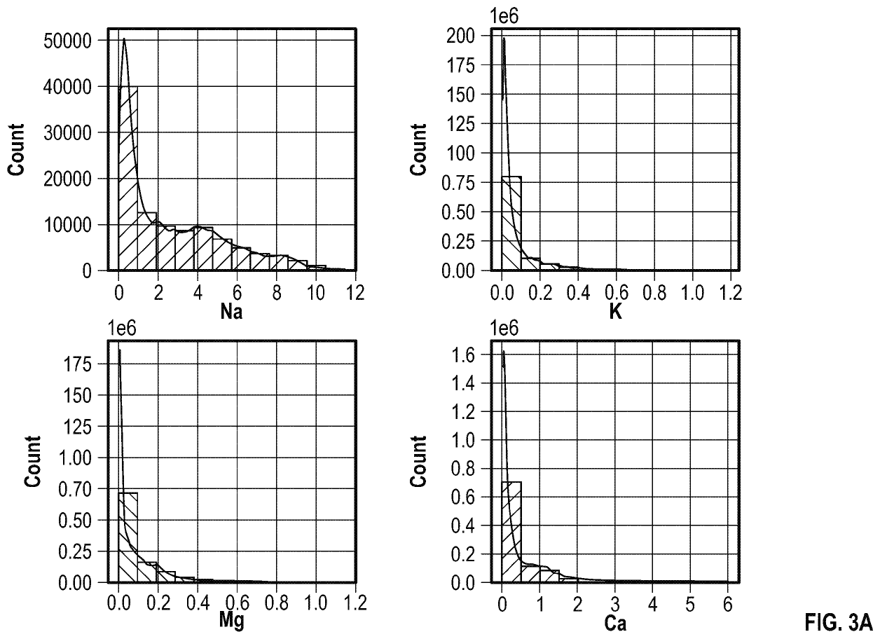

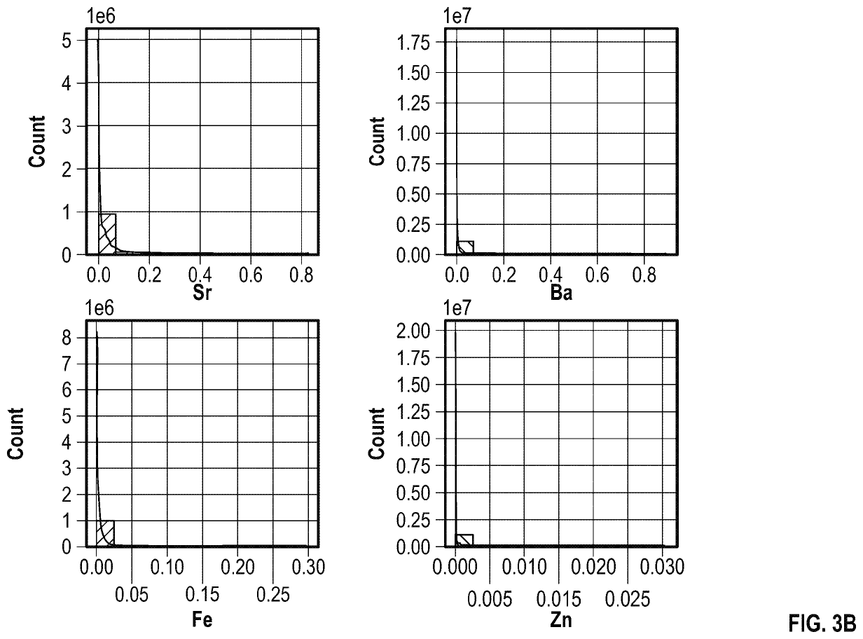

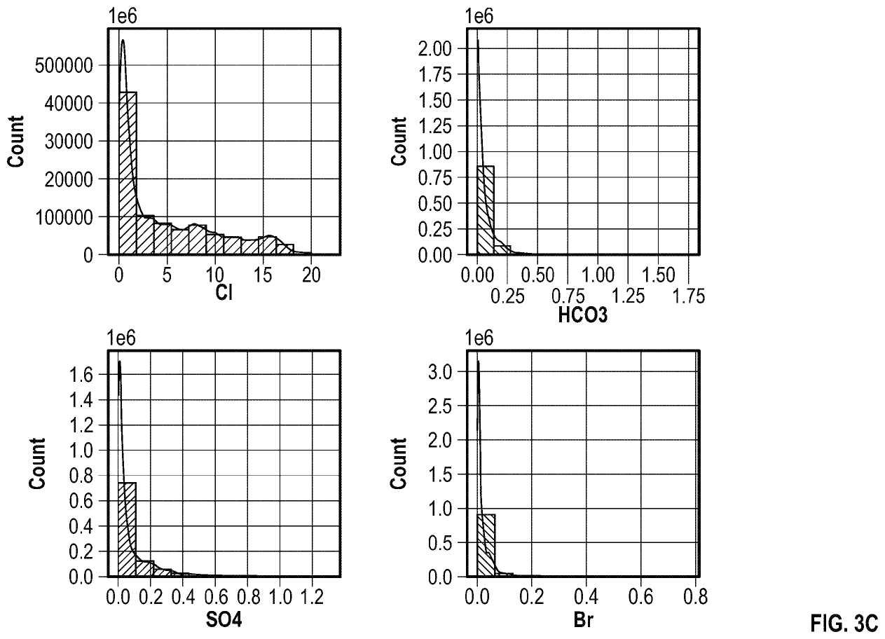

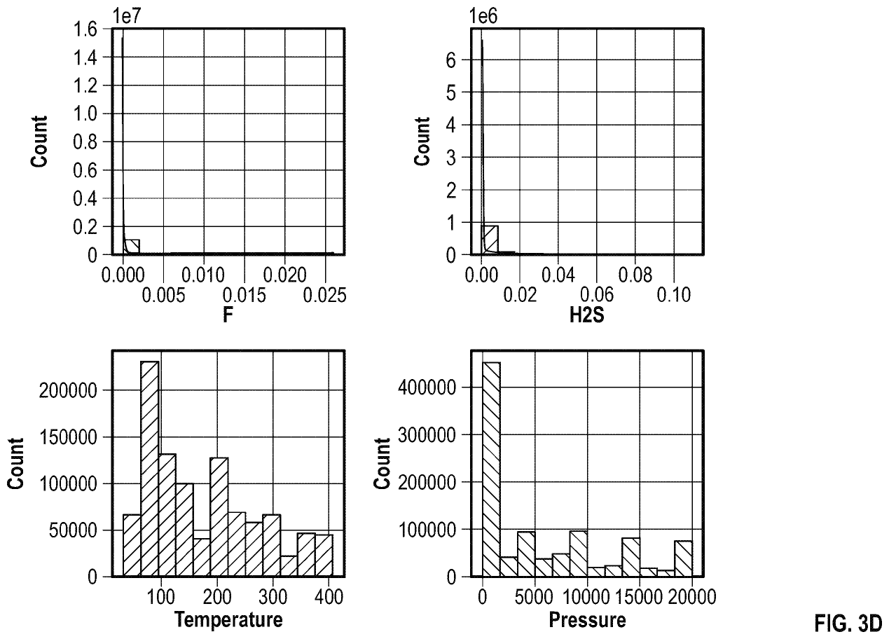

As the dataset only describes water composition, in some configurations, additional features, such as temperature (T) and pressure (p), can be added to cover broad ranges, such as 32-400 deg F. and 14.7-20,000 psi. These features can be varied either independently (within a grid) or simultaneously to cover the whole T-p space homogeneously, with the exception of combinations where waters were above the boiling point. Each water sample is subjected to ˜100 combinations of T and p conditions. Distributions of all 16 original features in the dataset are shown in FIG. 3.

### Example 3. Thermodynamic Simulations and Training Dataset

In some configurations, first principles thermodynamic simulations are performed with OLI Studio software (ver. 11.0 by OLI System, Inc.). In this example, water sample compositions were entered into the input field, and computational surveys for temperature and pressure were initiated. The OLI engine calculated over 500 outputs for each of 1,000,000 rows in the engineered produced water dataset. The outputs included: a) physicochemical properties, such as pH, hardness, total dissolved solids, densities, electrical conductivity, heat capacity, thermal conductivity, osmotic pressure, surface tension, viscosity, etc.; b) thermodynamic properties, such as enthalpy, entropy, and the Gibbs free energy; c) scaling and pre-scaling tendencies for numerous salts/minerals; and d) speciation of all components in the system, i.e., species present in liquid form, solids, and vapors.

Computation results were first saved as comma-separated value (csv) files for each sample, and then compiled into one large simulation results table. The table was further cleaned and processed, as non-converged simulations were removed, missing values were replaced (wherever appropriate), and saturation indices (SI) for key oilfield scaling minerals were calculated based on predicted pre-scaling tendencies. The list of processed scaling minerals is shown below:

| | | |
|---|---|---|
| CaSO₄ (Anhydrite) | SrCO₃ (Strontianite) | NaCl (Halite) |
| CaSO₄•0.5H₂O (Bassanite) | SrSO₄ (Celestine) | Mg(OH)₂ (Brucite) |
| CaSO₄•2H₂O (Gypsum) | BaSO₄ (Barite) | Fe(OH)₂ (Amakinite) |
| CaCO₃ (Aragonite) | FeCO₃ (Siderite) | |
| CaCO₃ (Calcite) | ZnS (Sphalerite) | |
| CaF₂ (Fluorite) | | |

The resulting table with ˜1,000,000 virtual experiment results and ˜500 computed properties was used to train ROMs.

### Example 4. Machine Learning Algorithm Screening

In a first round of screening, several machine learning (ML) models can be built using over 20 algorithms with default hyperparameters. The tests can be done with 10-fold cross-validation to ensure that models are not over-fitted. Mean values of key model metrics, such as mean absolute error (MAE), root mean square error (RMSE), and coefficient of determination (R2) can be used to compare models.

Depending on the target property, screening results varied, as, for example, liquid density can be predicted with reasonable accuracy (MAE of 4.4 kg/m³, which is less than 0.5% relative error) even with a simple multilinear regression model (see Table 2). More sophisticated algorithms can predict target properties better; however, the models might be more expensive to train, larger in size, and require more time for inference. All of these considerations are important for the final model selection and practical applications.

TABLE 2
Top 10 ML algorithm screening results (average values of 10
folds) for water density, with errors reported in kg/m³.

| Model | MAE | RMSE | R² |
|---|---|---|---|
| Extra Trees | 0.23 | 0.51 | 1.000 |
| Random Forest | 0.38 | 0.74 | 1.000 |
| CatBoost | 0.43 | 0.60 | 1.000 |
| Decision Tree | 0.62 | 1.21 | 1.000 |
| Extreme Gradient | 1.36 | 1.89 | 0.999 |
| Boosting | | | |
| Light Gradient | 1.43 | 1.96 | 0.999 |
| Boosting Machine | | | |
| MLP | 1.46 | 2.12 | 0.999 |
| Gradient Boosting | 2.30 | 3.07 | 0.998 |
| Ridge | 4.38 | 5.93 | 0.994 |
| Linear | 4.38 | 5.93 | 0.994 |

It must be emphasized that even liquid density is not always a simple property to predict based on original water composition, as the system can contain several phases, with precipitates (and/or vapors), and formation inevitably changes the liquid phase composition, and therefore its density. Prediction of precipitated solids (scale-forming minerals) and their saturation-indices is a more challenging task, particularly when more than one solid is formed. In some configurations, first principle thermodynamic simulators are used to predict such situations. In other configurations, ML-based surrogate models might do the same.

The algorithm screening results for saturation index of anhydrite are presented in Table 3. The saturation index is calculated as

$$SI = {log}_{10} ( \frac{TAP}{{K}_{sp}} ) , \qquad ( 2 )$$

where IAP is ionic activity product and Kₛₚ is the solubility product constant of a salt. The under-logarithm fraction under non-equilibrium conditions is referred to in the OLI software as the calculated pre-scaling tendency property.

TABLE 3
Top 10 ML algorithm screening results (average values
of 10 folds) for saturation index of anhydrite (CaSO₄).

| Model | MAE | RMSE | R² |
|---|---|---|---|
| Extra Trees | 0.011 | 0.023 | 1.000 |
| CatBoost | 0.018 | 0.024 | 1.000 |
| Random Forest | 0.018 | 0.035 | 0.999 |
| Decision Tree | 0.029 | 0.058 | 0.998 |
| Extreme Gradient | 0.047 | 0.062 | 0.998 |
| Boosting | | | |
| Light Gradient | 0.051 | 0.068 | 0.997 |
| Boosting Machine | | | |
| Gradient Boosting | 0.091 | 0.122 | 0.991 |
| AdaBoost | 0.282 | 0.356 | 0.925 |
| Ridge | 0.571 | 0.734 | 0.679 |
| Linear | 0.572 | 0.734 | 0.679 |

As previously shown for liquid density, ML algorithms based on gradient boosting and decision tree methods lead the pack with cross-validation average errors of 0.01-0.09 (MAE). For the SI anhydrite range in the training dataset of −8.3-2.2, the best absolute error (Extra Trees) converts to 0.1% relative error. This error is still an average of only 10 folds and is not validated on unseen data, so is likely optimistic. On the other hand, the screened algorithms are not yet optimized. Of note, the best-showing Extra Trees model can be very bulky, occupying several gigabytes of disk space, which is impractical.

### Example 5. Machine Learning Model Tuning and Validation

Example 4 demonstrated that physicochemical properties and scaling potential can be predicted with ML-based ROMs with reasonable accuracy. There are multiple ways, known to those skilled in the art of data science, to further optimize ML models, for example, by tuning algorithm hyperparameters, using more rigorous cross-validation techniques, etc.

The present example demonstrates how an ensemble model, which combines several weak models (learners), can be beneficial for ROM development.

In the present example, three gradient boosting techniques, such as extreme gradient boost (XGB), CatBoost and Light Gradient Boosting Machine (LGBM) were selected as initial models, based on screening results and practical consideration. The three models were combined or trained on multiple stratified folds using bagging and stack assembling techniques with an auto-ML library (“AutoGluon-Tabular: Robust and Accurate AutoML for Structured Data”). The model optimization performed on a powerful cluster allowed for a) reducing model over-fitting with a larger number of folds; and b) creating a model with SI anhydrite prediction errors of 0.02 (0.2%) on unseen validation dataset.

### Example 6. Surrogate Model Applications

Once the family of ROMs is trained and optimized for the target properties of interest, they can be used to predict water properties and/or scaling potential on-the-fly, wherever and whenever they are needed. In various embodiments or applications of the present disclosure, the models can be deployed in various digital workflows, e.g., hosted on a central server and called though an Application Programming Interface (API), used inside an end-user application (local or web-based), or even deployed near the data source, using Edge computing technologies, for example, coupled with analytical instruments or sensors, which provide water composition. The ROMs can be used to enhance (e.g., speed up or optimize) response to various controls or various processes, for example, chemical dosing, pump flow rate adjustment, pressure regulation, actuating of a valve, or adjustment of fluid temperature within equipment (for example, pipes, tanks, or other vessels).

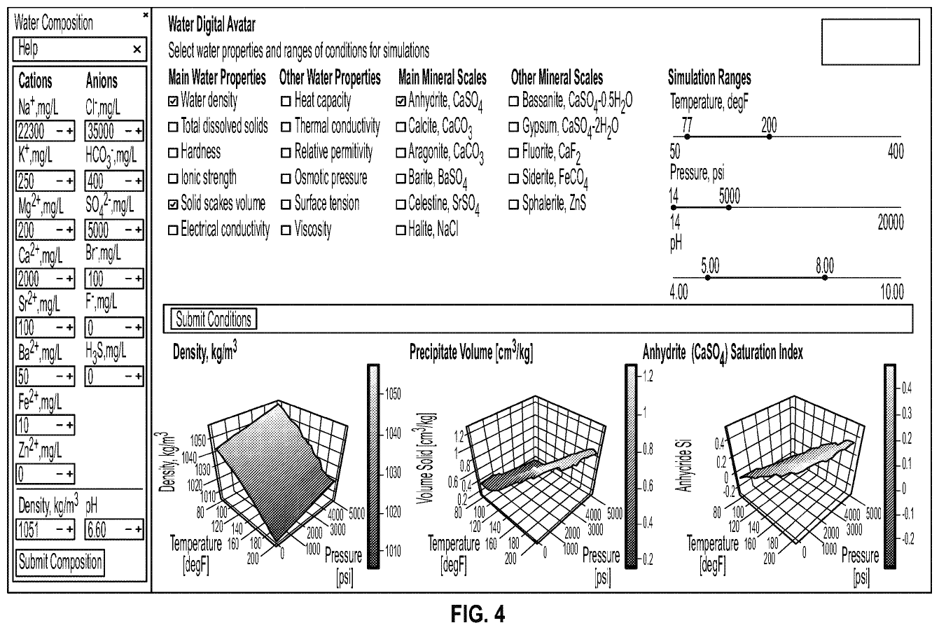

An example of a web-based predictor according to the disclosure is shown on FIG. 4, which relies on manual user input (left side bar) and computes several water properties on user's request, as a function of temperature and pressure.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. 112(f). However, for any claims containing elements designated in any other manner, it is intended that such elements are not to be interpreted under 35 U.S.C. 112(f).

1. A method of predicting properties of a chemical system, the method comprising:
generating a family of surrogate or reduced order models; and
training the family of surrogate or reduced order models on first principle simulation results.

2. The method of claim 1, wherein the predicted properties comprise one or more of physicochemical properties, thermodynamic properties, and phase speciation properties.

3. The method of claim 1, wherein the chemical system comprises water with dissolved species in contact with solid precipitates, gases, and other liquids.

4. The method of claim 3, wherein the other liquids comprise oil.

5. The method of claim 1, wherein the family of surrogate models is based on one or more machine learning algorithms.

6. The method of claim 1, further comprising constructing a training dataset from data subjected to data engineering steps.

7. The method of claim 6, wherein the data engineering steps comprise one or more of sampling, feature engineering and pre-processing, outlier removal, and target transformations.

8. The method of claim 1, further comprising using the surrogate models as stand-alone simulators, incorporating the surrogate models into user applications, or deploying the surrogate models on edge devices on or near sensors and/or analytical instruments.

9. The method of claim 8 further comprising using multiple simulators to form a range of family of results.

10. The method of claim 9 wherein the range of family of results comprises statistical evaluation of quality of the surrogate models result such as confidence interval or min/max/avg.

11. The method of claim 10 further comprising using the surrogate models to predict deviations or potential errors in one or more simulators.

12. The method of claim 1 further comprising using the surrogate models to speed up reaction time of a response to control or to optimize a process.

13. The method of claim 12 wherein the process comprises dosing of a chemical, adjustment of a pump flow rate, regulation of a pressure, adjustment of fluid temperature within equipment, or actuating a valve.

14. A method of predicting physicochemical properties of a chemical system, the method comprising:
training and optimizing reduced order models (ROMs) for one or more target properties using one or more machine learning models; and
predicting the physicochemical properties of the chemical system using the trained and optimized ROMs.

15. The method of claim 14, further comprising:
screening multiple machine learning models for the one or more target properties using model metrics to compare models; and
selecting one or more of the machine learning models based on the model metrics,
wherein training and optimizing the ROMs comprising training and optimizing the ROMs using the one or more machine learning models selected based on the model metrics.

16. The method of claim 14, wherein the chemical system comprises produced water.

17. The method of claim 14, further comprising performing principal component analysis on a representative dataset to produce an engineered dataset.

18. The method of claim 17, further comprising using the engineered dataset to train the ROMs.

19. The method of claim 14, further comprising deploying the trained and optimized ROMs in one or more digital workflows.

20. The method of claim 19, wherein the one or more digital workflows comprise optimizing one or more processes, the one or more processes comprising one or more of dosing of a chemical, adjustment of a pump flow rate, regulation of a pressure, adjustment of fluid temperature within equipment, and actuating a valve.
